(19) United States
(12) Patent Application Publication
(10) Pub. No.: US 2019/0189252 A1
(43) Pub. Date: Jun. 20, 2019

(54) CORRELATING HEALTH OUTCOMES WITH VALUES OF VARIABLES IN MANAGEMENT PROTOCOLS OF PATIENTS

(71) Applicant: Medtronic, Inc., Minneapolis, MN (US)

(72) Inventors: Jennifer L. Milbrandt, Excelsior, MN (US); Lindsay M. Streeter, Waconia, MN (US); Jennifer K. Bravinder, Denham Springs, LA (US); Jeffrey D. Chismar, Plymouth, MN (US); Jeffrey D. Naslund, Waconia, MN (US)

(21) Appl. No.: 15/841,686

(22) Filed: Dec. 14, 2017

(51) Int. Cl. G16H 10/60; G16H 80/00

(52) U.S. Cl. CPC G16H 10/60; G16H 15/00; G16H 80/00

(57) ABSTRACT

A computing system receives an indication of user input indicating whether a clinician accepts use of a set of default values for variables in a management protocol (MP) template in an MP for a particular patient. The computing system generates a description of the MP for the particular patient that specifies the values of the variables in the MP for the particular patient. Additionally, the computing system provides the description of the MP for the particular patient to the particular patient. Subsequently, the computing system receives data indicating health outcomes of the particular patient. The computing system generates, based on the health outcomes of patients, comparison data comparing health outcomes of patients whose MPs specify a first set of values of the variables versus health outcomes of patients whose MPs specify a second, different set of values of the variables.

### BACKGROUND

Clinicians frequently provide follow-up action descriptions relating to their patients. For example, if a patient sees a physician for an ear infection, the clinician may include an action description that advises the patient to take 2 antibiotic pills per day for 14 days and avoid drinking alcohol for two weeks. Other types of follow-up action descriptions may be significantly more complex. For instance, the follow-up action description given to a patient after heart surgery may be very lengthy and include information regarding how long to avoid driving a vehicle, how much water to drink each day, and so on.

To assist clinicians, computing systems have been developed that provide generic follow-up action descriptions to patients. For example, all patients with the ear infection may be given the same follow-up action descriptions. Such computing systems may provide the follow-up action descriptions to patients in various ways. For example, a computing system may generate a message providing the same follow-up action descriptions for all patients. The computing systems may print out the follow-up action descriptions, email the follow-up action descriptions, or otherwise provide the follow-up action descriptions to patients.

However, such computing systems present problems to clinicians because they lack flexibility for clinicians to customize the follow-up action descriptions given to individual patients. Thus, to customize the follow-up action descriptions given to an individual patient, a clinician may manually remove text snippets of the generic follow-up action descriptions and write in alternative follow-up action descriptions. For example, in the ear infection example given above, the clinician may remove a text snippet about taking 2 antibiotic pills for 14 days and manually write in 2 antibiotic pills for 21 days.

### SUMMARY

This disclosure describes techniques relating to improvements to computing systems that generate data reporting health outcomes of use of different values for variables in follow-up action descriptions. As described herein, a variable includes a specific follow-up action description that contains a space filled by the value of the variable. There are various technical challenges associated with existing computing systems for providing follow-up action descriptions, such as those described above. For example, it is desirable to determine whether the follow-up action descriptions given to patients lead to positive health outcomes for the patients and to determine whether the follow-up action descriptions are in fact necessary. While a computing system may collect data regarding the health outcomes of patients, it may be difficult for the computing system to collect statistically significant data correlating health outcomes of patients to particular sets of follow-up action descriptions if clinicians are routinely customizing the follow-up action descriptions in ways that are not tracked by the computing system when the computing system issues the follow-up action descriptions to the patients. At the same time, requiring clinicians to manually input their otherwise handwritten customized follow-up action descriptions into a computing system may be time consuming, and therefore may be skipped by busy clinicians. Moreover, it may be difficult for the computing system to collect statistically significant data if there are too few patients that are given the same follow-up action descriptions because clinicians are customizing the follow-up action descriptions. Thus, improvements may be needed to the computing system in order to ensure that the computing system is able to generate meaningful data regarding health outcomes correlated with follow-up action descriptions.

Techniques of this disclosure may provide solutions to this technical problem. For instance, as described in this disclosure, the computing system provides clinicians with the option of using default values for individual follow-up action descriptions for patients. A clinician may choose to follow the default values or select customized values for individual follow-up action descriptions. A clinician or group of clinicians may change the default values based on health outcomes of patients such that clinicians may use the default values for follow-up action descriptions for future patients. It is expected that clinicians will choose the default values in most circumstances, especially because the computing system allows the default values to be changed from time-to-time based on experience. Accordingly, most patients treated by a clinician or group of clinicians will receive the same follow-up action descriptions, thereby increasing the sample size of patients with the same follow-up action descriptions, while also providing the flexibility to provide and record customized values for follow-up action descriptions if needed. Moreover, different clinicians or groups of clinicians may have different default values for follow-up action descriptions. Thus, by providing the option to clinicians to use default values, the computing system may increase the sample sizes of patients receiving the same values for follow-up action descriptions, thereby improving the performance of the computing system. For example, techniques described in this disclosure may improve the way in which the computing system operates in generating accurate information regarding health outcomes correlated with different sets of follow-up action descriptions.

In one aspect, this disclosure describes a method for increasing accuracy of a computing system in correlating values of variables in management protocols with health outcomes, the method comprising: receiving, by a computing system, an indication of user input indicating whether a clinician accepts use of default values for a set of one or more variables in a management protocol (MP) template as values of the variables in an MP for a particular patient based on the MP template, the MP for the particular patient being a protocol the particular patient is advised to follow as part of the particular patient managing a medical condition, each of the variables including a respective action description that contains a space filled by the value of the variable; generating, by the computing system, a description of the MP for the particular patient, the description of the MP for the particular patient specifying the values of the variables in the MP for the particular patient; providing, by the computing system, the description of the MP for the particular patient to the particular patient; after providing the description of the MP for the particular patient to the particular patient, receiving, by the computing system, data indicating health outcomes of the particular patient; generating, by the computing system, based on the health outcomes of the particular patient and health outcomes of one or more additional patients managing the same medical condition, comparison data comparing health outcomes of patients whose MPs specify a first set of values of the variables versus health outcomes of patients whose MPs specify a second, different set of values of the variables; outputting, by the computing system, the comparison data for display; and after outputting the comparison data for display, receiving, by the computing system, an indication of user input indicating updates to the default values for the variables in the MP template.

In another aspect, this disclosure describes a computing system comprising: one or more processing circuits configured to: receive an indication of user input indicating whether a clinician accepts use of default values for a set of one or more variables in a management protocol (MP) template as values of the variables in an MP for a particular patient based on the MP template, the MP for the particular patient being a protocol the particular patient is advised to follow as part of the particular patient managing a medical condition, each of the variables including a respective action description that contains a space filled by the value of the variable; generate a description of the MP for the particular patient, the description of the MP for the particular patient specifying the values of the variables in the MP for the particular patient; provide the description of the MP for the particular patient to the particular patient; after providing the description of the MP for the particular patient to the particular patient, receive data indicating health outcomes of the particular patient; generate, based on the health outcomes of the particular patient and health outcomes of one or more additional patients managing the same medical condition, comparison data comparing health outcomes of patients whose MPs specify a first set of values of the variables versus health outcomes of patients whose MPs specify a second, different set of values of the variables; output the comparison data for display; and after outputting the comparison data for display, receive an indication of user input indicating updates to the default values for the variables in the MP template.

In another aspect, this disclosure describes a non-transitory computer-readable data storage medium having instructions stored thereon that when executed cause one or more processing units to: receive an indication of user input indicating whether a clinician accepts use of default values for a set of one or more variables in a management protocol (MP) template as values of the variables in an MP for a particular patient based on the MP template, the MP for the particular patient being a protocol the particular patient is advised to follow as part of the particular patient managing a medical condition, each of the variables including a respective action description that contains a space filled by the value of the variable; generate a description of the MP for the particular patient, the description of the MP for the particular patient specifying the values of the variables in the MP for the particular patient; provide the description of the MP for the particular patient to the particular patient; after providing the description of the MP for the particular patient to the particular patient, receive data indicating health outcomes of the particular patient; generate, based on the health outcomes of the particular patient and health outcomes of one or more additional patients managing the same medical condition, comparison data comparing health outcomes of patients whose MPs specify a first set of values of the variables versus health outcomes of patients whose MPs specify a second, different set of values of the variables; output the comparison data for display; and after outputting the comparison data for display, receive an indication of user input indicating updates to the default values for the variables in the MP template.

The details of one or more examples of the techniques are set forth in the accompanying drawings and the description below. Other features, objects, and advantages of the techniques will be apparent from the description, drawings, and claims.

### BRIEF DESCRIPTION OF DRAWINGS

FIG. 1 is a conceptual diagram illustrating an example environment in which techniques of this disclosure may be implemented.

FIG. 2 is a flowchart illustrating an example operation of a computing system in accordance with one or more techniques of this disclosure.

FIG. 3 is a flowchart illustrating an example operation for increasing an accuracy of a computing system in correlating values of variables in management protocols with health outcomes, in accordance with a technique of this disclosure.

FIG. 4 is a block diagram of an example configuration of an inventory management computer system that may be configured to implement the techniques of this disclosure.

### DETAILED DESCRIPTION

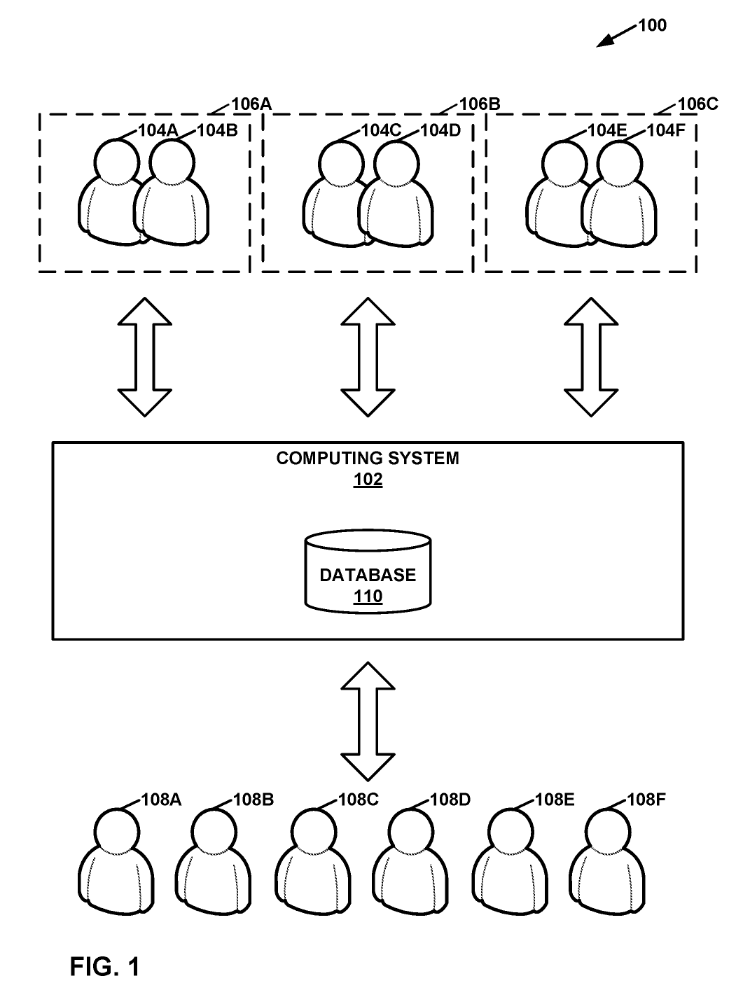

FIG. 1 is a conceptual diagram illustrating an example environment 100 in which techniques of this disclosure may be implemented. In the example of FIG. 1, environment 100 comprises a computing system 102. As described herein, computing system 102 enables clinicians to specify management plans for patients experiencing a medical condition and may provide the management plans to the patients. Additionally, computing system 102 may receive feedback information from the patients, evaluate the feedback to produce comparison data, and may allow the clinicians to modify default values in their management plans.

Computing system 102 may be implemented in various ways. For example, computing system 102 may comprise one or more server devices, personal computers, server blades, or other types of computing devices. Although not illustrated in the example of FIG. 1 for the sake of simplicity, computing system 102 may communicate (e.g., via one or more communication networks, such as the Internet) with one or more additional computing devices used by clinicians, patients, researchers, or other types of individuals. The additional computing devices may include personal computers, tablet computers, smartphones, laptop computers, and other types of devices.

Computing system 102 may comprise a database 110 configured to store data. Database 110 may be implemented in various ways. For example, database 110 may be implemented as one or more file systems, relational databases, or one or more other structured systems for storing data.

The example of FIG. 1 shows computing system 102 being able to communicate with a set of clinicians 104A-104F (collectively, “clinicians 104”). Although FIG. 1 shows six clinicians, techniques of this disclosure may be performed using more or fewer clinicians. The set of clinicians 104 may physicians, nurse practitioners, chiropractors, surgeons, or other types of medical professionals.

In the example of FIG. 1, clinicians 104 are divided into a set of groups 106A-106C (collectively, “groups 106”). Although FIG. 1 shows three groups, techniques of this disclosure may be performed using more or fewer groups. In different examples, groups 106 are defined in different ways. For example, each of groups 106 may correspond to a different hospital, clinic, healthcare provider network, or group thereof. For instance, group 106A may correspond to clinicians practicing in a first hospital, group 106B may correspond to clinicians practicing in a second hospital, and so on.

Additionally, the example of FIG. 1 shows computing system 102 being able to communicate with a set of patients 108A-108F (collectively, “patients 108”). Although FIG. 1 shows six patients, techniques of this disclosure are anticipated to be performed with many more patients. Each of patients 108 is experiencing or has experienced the same medical condition or event. For example, each of patients 108 may have undergone a knee replacement, each of patients 108 may have a particular type of implanted medical device, a particular type of cancer or infection, a particular type of diabetes, a particular type of injury, or another type of medical condition. Techniques of this disclosure may be used with any disease or medical condition.

After a patient 108 has visited a clinician 104, the clinician typically gives the patient follow-up action descriptions as part of a plan to help the patient manage and/or recover from the medical condition. For example, if the patient has had heart surgery, the clinician may give the patient an action description regarding how long the patient should avoid driving, an action description regarding anticoagulant use, an action description regarding whether to avoid weight-bearing activities, an action description regarding how the patient is to care for an incision, an action description regarding whether the patient should lie prone, and so on. There may be different sets of action descriptions for different medical conditions.

Moreover, different clinicians are known to use different follow-up action descriptions. For example, for patients recovering from a particular type of heart surgery, one clinician may instruct the patient not to drive for 6 weeks and another clinician may instruct the patient not to drive for 8 weeks. Variability among follow-up action descriptions may be greater between clinicians in different groups.

In accordance with a technique of this disclosure, computing system 102 may help clinicians 104 specify management plans for patients experiencing a medical condition. For example, computing system 102 may store (e.g., in database 110) management plan (MP) templates corresponding to different medical conditions. For example, one MP template may correspond to a first medical condition, a second MP template may correspond to a second medical condition, and so on. Each MP template includes a set of variables. Each of the variables includes a different action description that contains a space filled by the value of the variable. Example variables may include action descriptions regarding physical activity, walking, showering/bathing, work, driving, incisional care, follow-up appointments, equipment types, abductor pillows, fluid intake, physical therapy, weight bearing activities, pivoting/bending, flexion/extension restrictions, lying prone, anticoagulant use, and so on.

Each of the variables has an associated value. For example, a variable may correspond to the action description “X weeks without driving.” In this example, X is the space filled by the value of the variable. In other words, the value of the variable may be inserted instead of “X.” Thus, one clinician may use the value 6 as the value of this variable to indicate 6 weeks without driving and another clinician may use the value 8 as the value of this variable to indicate 8 weeks without driving. If the variable is unused in an MP, the variable may be considered to have a null value.

Database 110 of computing system 102 may store global default values for the variables in an MP template. The global default values may be the same for all clinicians using computing system 102. Database 110 of computing system 102 may also store default values of the variables in the MP template selected by individual clinicians (i.e., clinician-level default values) or groups of clinicians (i.e., group-level default values). For example, database 110 may store a global default value of 6 for the variable “X weeks without driving,” database 110 may store a group-level default value of 7 for this variable for group 106A, and database 110 may store a group-level default value of 5 for this variable for group 106B.

When a clinician is preparing an MP for a particular patient experiencing a particular medical condition, computing system 102 presents the clinician with an option of using default values for the variables in the MP template for the particular medical condition. Computing system 102 uses information about the particular patient to generate an MP template that contains a set of patient action descriptions for consideration by the clinician. The clinician may accept, decline, or modify the suggested action descriptions based upon their personal judgement and expertise. The suggested patient action descriptions can be based upon underlying clinical research and data analysis, which can be included as part of the presentation to the clinician. In this context, the MP template can provide educational purposes in addition to reinforcing the care plan. An MP template used in this manner contains only considerations for a clinician, rather than medical instructions, or prescriptions, directly to patients. For ease of discussion, the term “action descriptions” is used herein to denote both the action descriptions provided for consideration by a clinician and those action descriptions presented to a patient at the direction of a clinician.

The action descriptions are not limited to descriptions of actions to be carried out by the patient. For example, an action description could specify that a computer system (e.g., as part of a remote care management system) presents educational material to the patient. Such an action description could specify certain triggers for when the educational material is to be presented. Other, non-limiting, examples of action descriptions include descriptions of actions such as initiating a consultation between the patient and a clinician or other healthcare support personnel, sending reminders to the patient regarding actions to take (e.g., take their blood pressure), or notifying the prescribing clinician in the event that certain conditions are met (e.g., the patient has not followed the assigned MP).

For example, computing system 102 may first present the clinician with an option of using global default values or group-level default values. If the clinician declines, computing system 102 may present the clinician with an option of using clinician-level default values or other custom values. If computing system 102 receives an indication of user input to accept the default values of the variables in the MP template, computing system 102 may generate an MP for the particular patient that uses the default values of the variables. Computing system 102 may then provide the MP for the particular patient to the particular patient (e.g., via email, secure message center, postal mail, paper printout, etc.)

If computing system 102 receives an indication that the clinician does not want to use the default values for the variables in the MP template for the particular medical condition, computing system 102 may present to the clinician a user interface for selecting custom values for the variables in the MP template for the particular medical condition. For example, the default value for the variable “X weeks without driving” may be 8, but the clinician may choose 10 (resulting in the following follow-up action description of “10 weeks without driving) based on the clinician's judgment and knowledge regarding the particular patient. In another example, the default value for the variable “X weeks of anticoagulant use” may be null (meaning the text snippet for the variable is not included in the patient's MP), but the clinician may set the value to a non-null number based on the clinician's knowledge that the patient has a history of blood clots. However, it is expected that clinicians will frequently use the default values because doing so may simply be easier than selecting custom values. If computing system 102 receives an indication of user input to select a custom value of an variable of the MP template, computing system 102 may generate an MP for the particular patient that uses the custom value of the variable.

Computing system 102 may then provide a description of the MP for the particular patient to the particular patient. The description of the MP for the particular patient may comprise text snippets (i.e., predetermined text) corresponding to values of the variables. For example, a text snippet in the description of the MP may indicate “10 weeks without driving.”

After a patient (e.g., one of patients 108) has received the description of the MP for the patient, computing system 102 may receive and store data indicating health outcomes of the patient. For example, computing system 102 may receive indications of user input indicating the health outcomes of the patient. For instance, in this example, computing system 102 may present a user interface that allows the patient, clinician, or other person to input the data indicating the health outcomes of the patient. Thus, computing system 102 may receive the data in the form of survey answers. In some examples, computing system 102 may receive the data from electronic medical records of the patient.

The health outcomes of a patient may include changes in the health status of the patient which are attributable to a planned intervention or series of interventions, regardless of whether such an intervention was intended to change health status. For example, occurrence of blood clots may be a health outcome attributable to a knee replacement surgery (i.e., an intervention). In another example, relief of knee pain may be another health outcome attributable to a knee replacement surgery.

Furthermore, computing system 102 may generate, based on the health outcomes of one or more patients 108 managing the same medical condition, comparison data comparing health outcomes of patients whose MPs specify a set of values of the variables versus health outcomes of patients whose MPs specify a different set of values of the variables. For example, a first set of patients (e.g., patients 108A, 108B, and 108C) may receive MPs specifying a first value for a particular variable and a second set of patients (e.g., patients 108D, 108E, and 108F) may receive MPs specifying a second, different value for the particular variable. In practice, the sets of patients may include far larger numbers of patients. In this example, computing system 108 may compare health outcomes for the first set of patients and the second set of patients to determine whether the incidence of particular health outcomes differs for patients given MPs having variables with different values.

Computing system 102 may output the comparison data for display to the clinician. For example, computing system 102 may output for display a user interface comprising statistics regarding whether individual health outcomes are more or less likely to occur in patients given MPs with variables having different values. For instance, the user interface may indicate that incidence of motor vehicle crashes (a health outcome) is the same in patients receiving MPs that indicate no driving for 5 weeks versus MPs that indicate no driving for 6 weeks. In this example, the user interface may further indicate that the incidence of motor vehicle crashes is higher in patients receiving MPs that indicate no driving for 4 weeks.

After outputting the comparison data for display to the clinician, computing system 102 may receive an indication of user input indicating updates, if any, from the clinician to the default values for the variables in the MP template. For instance, in the example above regarding motor vehicle crashes, a clinician or group of clinicians may modify the value for the “no driving for X weeks” parameter from 6 to 5, so as not to unnecessarily prevent patients from driving for an extra week. Computing system 102 may use the updated default values in future MPs.

In this way, computing system 102 may receive an indication of user input indicating whether a clinician 104 accepts use of a set of default values for variables in a MP template as values of the variables in an MP for a particular patient based on the MP template. In some examples, prior to receiving the indication of user input indicating whether the clinician accepts the use of the set of default values, computing system 102 presents a list of the default values for the set of variables in the MP template for the patients to follow as part of the patients managing the medical condition. The MP for the particular patient is a protocol for the particular patient to follow as part of the particular patient managing the medical condition. The set of variables includes one or more variables. Each of the variables may correspond to a different action description that contains a space filled by the value of the variable. The action description with the filled space may be provided to patients in the form of an instruction, a recommendation, a piece of advice, or a piece of information for patient consideration. In some examples, an action description may be provided to a remote monitoring service to control how the remote monitoring service interacts with a patient. For instance, a variable may indicate a frequency with which the remote monitoring service asks the patient for a particular type of information.

Computing system 102 may generate a description of the MP for the particular patient. The description of the MP for the particular patient specifies the action descriptions with filled-in values of one or more variables in the MP for the particular patient. Furthermore, computing system 102 may provide the description of the MP for the particular patient to the particular patient. After providing the description of the MP for the particular patient to the particular patient, computing system 102 may receive data indicating health outcomes of the particular patient. Computing system 102 may generate, based on the health outcomes of the particular patient and health outcomes of one or more additional patients managing the same medical condition, comparison data comparing health outcomes of patients whose MPs specify a first set of values of the variables versus health outcomes of patients whose MPs specify a second, different set of values of the variables. Additionally, computing system 102 may output the comparison data for display. After outputting the comparison data for display, computing system 102 may receive an indication of user input indicating updates to the default values for the variables in the MP template.

In some examples, patients 108 are enrolled in a remote monitoring service. A patient may be enrolled in the remote monitoring service at the time the patient is discharged from a clinical setting. The patient may be provided with their management protocol, including follow-up action descriptions, as part of being discharged. The remote monitoring service may be designed to reduce readmission of patients 108. Patients enrolled in the remote monitoring service may provide information to a computing system (e.g., computing system 102 or a different computing system) as part of their individual management protocols. For example, a patient may provide information to the computing system daily for the first seven days, every three days for days 8-21 and every four days for days 22-30 after a medical intervention. In some examples, the remote monitoring service provides equipment (e.g., computers, tablets, mobile devices, etc.) to the patients to allow patients 108 to communicate with the computing system. Information provided by patients 108 may include biometric data, symptom data, information indicating health outcomes of patients 108, and so on. Additionally, clinical staff supporting the remote monitoring service may review and assess the information sent by the patients. If needed, the clinical staff may communicate with individual patients to gather clarifying information and/or to educate the patients on their medical conditions in accordance with their management protocol. If needed, the clinical staff may provide reports regarding individual patients to the patients' clinicians to determine if a change in medication or care plan is needed.

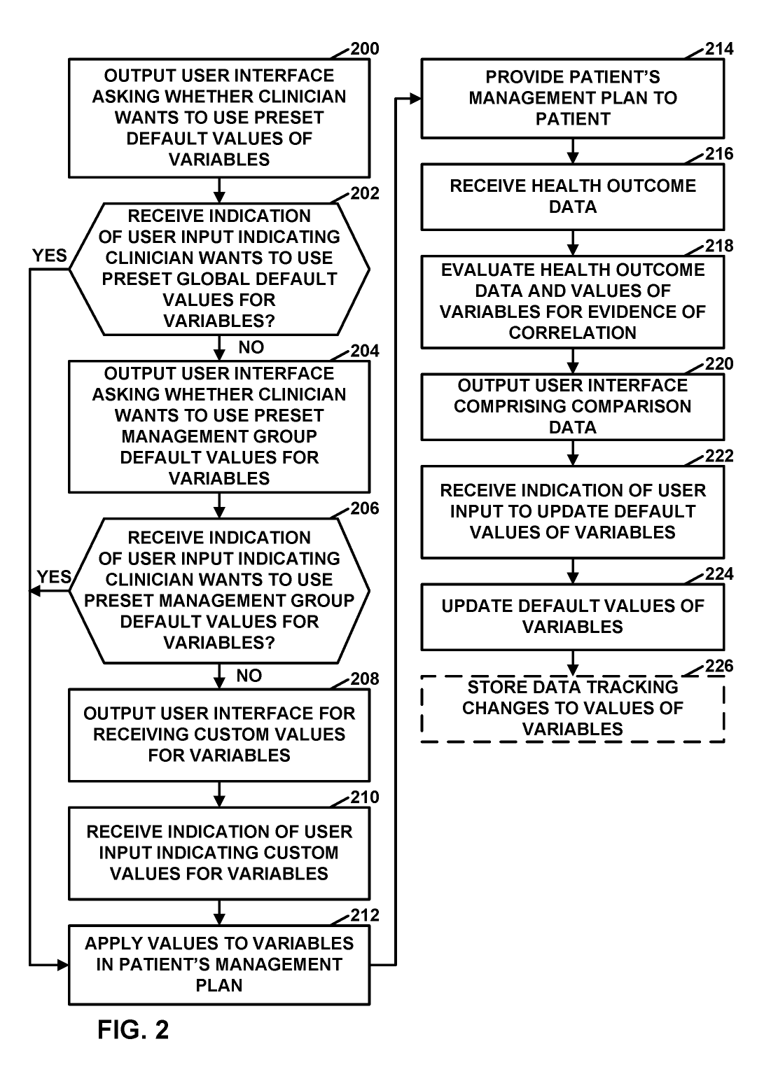

FIG. 2 is a flowchart illustrating an example operation of a computing system in accordance with one or more techniques of this disclosure. The flowcharts of this disclosure are provided as examples. Other examples in accordance with techniques of this disclosure may include, more, fewer, or different actions or actions may be performed in different orders.

In the example of FIG. 2, computing system 102 may output for display a user interface asking whether a clinician wants to use preset global default values of variables in a patient's MP (200). Subsequently, computing system 102 may receive an indication of user input indicating whether the clinician wants to use preset global default values for variables in the patient's MP (202).

In response to receiving an indication of user input indicating that the clinician does not want to use the preset global default values for the variables (“NO” branch of 202), computing system 102 may output for display a user interface asking whether the clinician wants to use preset management group default values (i.e., group-level default values) for variables in the patient's MP (204). The group-level default values presented to the clinician may be specific to a group to which the clinician belongs. For instance, in the example of FIG. 1, computing system 102 may present to clinicians 104A and 104B group-level default values for group 106A, computing system 102 may present to clinicians 104C and 104C group-level default values for group 106B, and so on.

Subsequently, computing system 102 may receive an indication of user input indicating whether the clinician wants to use the preset management group default values for the variables in the patient's MP (206). Different groups of clinicians (i.e., “management groups” may have different default values. This may be desirable because it allows for diversity among values of variables. Additionally, it allows differently situated clinician groups to adapt default values based on local needs. For example, a first clinician group may be located in an urban area and a second clinician group may be located in a rural area. In this example, because it may be more difficult to travel to a clinician visit in a rural area, the default value used by the second clinician group for recurring follow-up visits may indicate less frequent visits than for the first clinician group.

Thus, computing system 102 may receive an indication of user input indicating whether a second clinician in a second group of clinicians accepts use of a set of group-level default values specific to the second group of clinicians as values of the variables in an MP for a second patient based on the MP template. The set of group-level default values specific to the second group of clinicians may have one or more values different from values of the variables in the first set of group-level default values.

In response to receiving an indication of user input indicating that the clinician does not want to use the preset management group default values for variables in the patient's MP (“NO” branch of 206), computing system 102 may output a user interface for receiving custom values for variables in the patient's MP (208). Subsequently, computing system 102 may receive indications of user input indicating custom values for variables (210). In some examples, the user interface for receiving the custom values for the variables in the patient's MP may include features for inputting the custom values for the variables. In such examples, the features may be automatically populated with default values (e.g., global-level default values, group-level default values, clinician-level default values, etc.) for the variables. Furthermore, in some examples, a clinician may have multiple sets of clinician-level default values from which the clinician may select. Thus, rather than manually change custom values in a particular way multiple times, the clinician may select in the user interface one of the sets of clinician-level default values.

In this way, based on the indication of user input indicating that the clinician does not accept use of the default values for the one or more variables in the MP template in the MP for a particular patient, computing system 102 may output, for display, a user interface comprising user interface features for selecting custom values for the one or more variables in the MP template in the MP for the particular patient. Computing system 102 may generate the description of the MP for the particular patient such that the description of the MP for the particular patient specifies the custom values for the variables in the MP for the particular patient.

In some examples, computing system 102 may check the custom values to determine whether the custom values fall within normal expectations. Performing this check may reduce the possibility of improper selection of custom values, and ensure an appropriate level of care.

Furthermore, in some examples, set of variables may be linked into groups. A clinician may be able to turn on or off a group of linked variables (i.e., set values of variables in the group to null) with a single input (e.g., clicking on a checkbox). Thus, computing system 102 may enable or disable a group of variables in one category.

In response to receiving an indication of user input indicating that the clinician wants to use the preset global default values for the variables in the patient's MP (“YES” branch of 202), in response to receiving an indication of user input indicating that the clinician wants to use the preset management group default values for the variables in the patient's MP (“YES” branch of 206), or after receiving the indications of user input indicating the custom values of the variables in the patient's MP (210), computing system 102 applies the values (i.e., the preset global default values, the preset management group default values, or the custom values) to the variables in the patient's MP (212). For example, computing system 102 may fill in values of certain variables in a template containing action descriptions (e.g., snippets of text) corresponding to the variables. In this example, if the value of an variable is null, computing system 102 may omit the snippet of text corresponding to the variable from the text of the patient's MP.

Furthermore, in the example of FIG. 2, computing system 102 may provide the patient's MP to the patient (214). Computing system 102 may provide the patient's MP to the patient in various ways. For example, computing system 102 may provide the patient's MP to the patient via email, may print out the patient's MP, generate a secure webpage containing the patient's MP, send a text, voice or video message containing the patient's MP, and so on. In some examples, computing system 102 may store, in database 110, data indicating a time and date when computing system 102 provided the patient's MP to the patient. Computing system 102 may use the data indicating the time and date to identify when the patient's MP is active. In some examples, computing system 102 uses particular variables in the patient's MP to configure devices used by patients as part of a remote monitoring program. For example, one variable of the patient's MP may be an action description that indicates a schedule with which the device is to request the patient submit a particular type of information. In this example, computing system 102 may use this variable to configure a device used by the patient to output, according to the schedule, health check messages that remind or request the patient to submit the particular type of information.

Subsequently, computing system 102 may receive health outcome data (216). The health outcome data may contain information regarding health outcomes of the patient. Computing system 102 may receive the health outcome data in various ways. For example, computing system 102 may output one or more webpages comprising survey questions. In this example, computing system 102 may receive the health outcome data in the form of answers to the survey questions. In some examples, computing system 102 may receive at least a portion of the health outcome data from electronic medical records of the patient. In some examples, the patient is provided a device (e.g., tablet computer, smartphone, etc.) for communicating with computing system 102. Computing system 102 may receive at least a portion of the information regarding the health outcomes via such a device provided to the patient. In some such examples, the device as one or more configurable device settings. For instance, the device settings may include a device setting that controls a frequency with which the device communicates action descriptions to computing system 102. Computing system 102 may set values of the device settings based on a patient's management protocol.

As briefly discussed above, in some examples, a device provided to a patient may generate health check messages. Each health check message may be associated with a schedule for how frequently the health check message is provided to the patient. This schedule may act as another variable of the patient's MP. Thus, there may be a default schedule for the health check message and a clinician may select a non-default schedule for the health check message. For example, if a patient is taking an antibiotic for an ear infection, the patient's MP may include a health check message that asks the patient a question about monitoring symptoms of ear drainage. In this example, the default schedule may ask the question every day. However, in this example, a clinician may set the schedule to every other day, or every day for the first week and every three days thereafter.

Furthermore, in the example of FIG. 2, computing system 102 may evaluate the health outcome data, along with health outcome data for other patients, and the values of the variables for evidence of correlation (218). In other words, computing system 102 may generate, based on the health outcomes of the patient and health outcomes of one or more additional patients managing the same medical condition, comparison data comparing health outcomes of patients whose MPs specify a first set of values of the variables versus health outcomes of patients whose MPs specify a second set of values of the variables that is different from the first set of values of the variables. For instance, the comparison data may compare health outcomes of patients whose MPs specify the default values for a first group of clinicians (i.e., first preset management group default values) to health outcomes of patients whose MPs specify the default values for a second group of clinicians (i.e., a second preset management group default values). In a particular example, computing system 102 may determine whether patients that have a longer restriction on a type of activity or are prescribed specific medications are less likely to have adverse events such as prehospitalization or exacerbation of symptoms. In some examples, as part of generating the comparison data, computing system 102 may generate data indicating whether an incidence of a positive health outcome is greater when the first set of values of the variables is used as opposed to the second set of values of the variables.

In some examples, computing system 102 uses machine learning techniques to generate the comparison data. For instance, computing system 102 may use a decision tree regressor, a random forest regressor, a ridge regressor, or another type of machine learning algorithm that generates predictions based on health outcome data and the values of the variables.

In generating the comparison data, computing system 102 may use data stored by computing system 102 regarding when MP's were issued to patients to help ensure that the data regarding health outcomes are applied on the same timelines. For instance, based on the dates when the MP's were issued to patients, computing system 102 may determine incidence of particular health outcomes within particular time periods, such as 6 months, 1 year, etc.

In some examples, as the patient goes through the management program for a medical condition, computing system 102 identifies where changes in a schedule or variables could be applied to determine if other interventions are more applicable in creating a positive outcome. For example, if a patient is being monitored for an ear infection with antibiotics and the clinician wants to ensure adherence to the treatment plan. The messaging could be daily to ask if the patient is taking the medications as directed. If the patient answers that the medications are being taken as directed, computing system 102 may, after a number of positive answers, decrease the question frequency to focus on prevention of ear infections instead of management of the acute systems either through notification to the clinician. If the patient is not taking medications as directed, computing system 102 may notify the clinician regarding non-adherence to change the questions or interventions to focus on why taking medications as directed even when feeling better is important to ensure the infection is fully resolved to prevent further symptoms or recurrence of the infection.

In the example of FIG. 2, computing system 102 may output for display a user interface comprising the comparison data (220). For example, computing system 102 may comprise a list of one or more health outcomes and the incidence of each of the listed health outcomes when patients are given MPs with variables with different values. Thus, a person (e.g., a clinician) reviewing the comparison data may be able to determine whether the default values should be adjusted such that patients have better health outcomes. In some examples, the comparison data may comprise a comparison of health outcomes of patients whose MPs had variables with custom values and health outcomes of patients whose MPs have variables with default values. Learning which values are more likely to have positive health outcomes may result in less clinician visits, causing a decrease in overall cost in health condition management.

Thus, subsequently, computing system 102 may receive one or more indications of user input to update default values of variables (222). For example, computing system 102 may output for display a user interface comprising features (e.g., text boxes, drop boxes, etc.) for editing values of variables. In this example, computing system 102 may receive the one or more indications of user input as data entered into such features. The updated default values may be the global default values (i.e., the preset global default values) applicable for all users of computing system 102, group-level default values (i.e., preset management group default values), or clinician-level default values, if used. Different users may have different permissions to change default levels. For instance, an individual clinician might not have permission to change system-level default values, but may be able to change group-level default values or clinician-level default values. Although not illustrated in the example of FIG. 2, computing system 102 may use other levels of default values in a similar manner. For instance, there may be regional-level default values at a level between global default values and group-level default values.

In some examples where clinician-level default values are used, and clinician-level default values are automatically populated into a user interface for providing custom values, computing system 102 may, in effect, automatically generate clinician-specific templates. In some examples, a clinician does not need to specifically update clinician-level default values. Rather, in such examples, computing system 102 may simply re-use the clinician's previously-selected custom values as the clinician's clinician-specific default values. Thus, computing system 102 may automatically set values in the user interface features to custom values previously selected by the clinician.

Additionally, in response to receiving the one or more indications of user input to update the default values of the variables, computing system 102 may update the default values of the variables (224). For example, computing system 102 may update database 110 to indicate the updated default values of the variables.

In some examples, computing system 102 may automatically update particular default values. For instance, computing system 102 may automatically update the default value of one variable based on an update to another variable. For example, if a patient is being treated for an ear infection and the default value for a variable regarding the duration of a medication treatment plan is changed to 21 days, computing system 102 may extend the default value for a variable regarding the number of days to monitor body temperature to 21 days, thereby ensuring appropriate biometric monitoring to allow for clinicians to be alerted if patients are still experiencing symptoms.

Furthermore, in some examples, computing system 102 may store (e.g., in database 110) data tracking changes to default values of variables and changes to values of variables in particular patients' MPs (226). Thus, the stored data may be used to review previous values of default values of the variables and values of variables in particular patients' MPs. Thus, the tracked changes to the default values may serve as a basis for system auditing to see when and what was changed for the values, as well as what each patient and/or group was assigned as measurements.

Furthermore, computing system 102 may store (e.g., in database 110) data tracking histories of interactions between patients and clinicians. Such data may be a source of information for computing system 102 to measure an individual patient's outcomes to specific applied interventions. Such data may also be used to conduct comparative analysis within health populations to understand what interventions works best for a specific type of patient. In addition, comparative analysis from such data could lead to additional understanding on how external factors such as socioeconomic, geographical and health literacy can affect the outcomes of interventions.

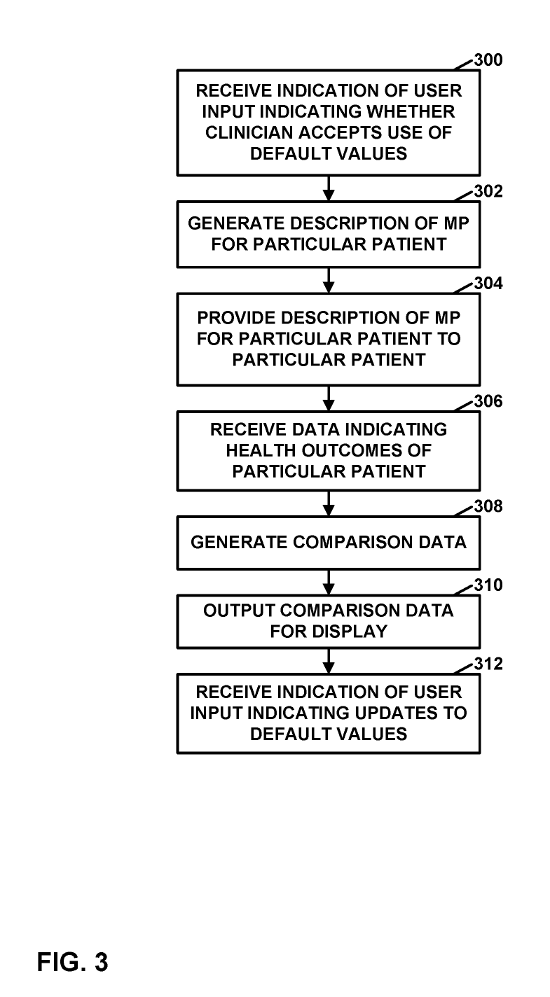

FIG. 3 is a flowchart illustrating an example operation for increasing accuracy of computing system 102 in correlating values of variables in management protocols with health outcomes. In the example of FIG. 3, computing system 102 may receive an indication of user input indicating whether a clinician accepts use of a set of default values for variables in a MP template as values of the variables in an MP for a particular patient based on the MP template (300). The MP for the particular patient is a protocol the particular patient is advised to follow as part of the particular patient managing a medical condition. The set of variables include one or more variables.

Furthermore, in the example of FIG. 3, computing system 102 may generate a description of the MP for the particular patient (302). The description of the MP for the particular patient specifies the values of the variables in the MP for the particular patient. The generated description of the particular patient's MP may include action descriptions for one or more of the variables in the particular patient's MP. In some examples, the generated description of the particular patient's MP does not include action descriptions for one or more variables in the particular patient's MP, such as those variables that configure computing systems and devices. Furthermore, computing system 102 may provide the description of the MP for the particular patient to the particular patient (304).

After providing the description of the MP for the particular patient to the particular patient, computing system 102 may receive data indicating health outcomes of the particular patient (306). Furthermore, computing system 102 may generate, based on the health outcomes of the particular patient and health outcomes of one or more additional patients managing the same medical condition, comparison data comparing health outcomes of patients whose MPs specify a first set of values of the variables versus health outcomes of patients whose MPs specify a second, different set of values of the variables (308).

Computing system 102 may output the comparison data for display (310). For example, computing system 102 may output the comparison data for display on a display screen used by the clinician. In some examples, computing system 102 may also output the comparison data for display on a display screen used by a patient. After outputting the comparison data for display, computing system 102 may receive an indication of user input indicating updates to the default values for the variables in the MP template (312). For example, computing system 102 may receive the indication of user input via a graphical user interface.

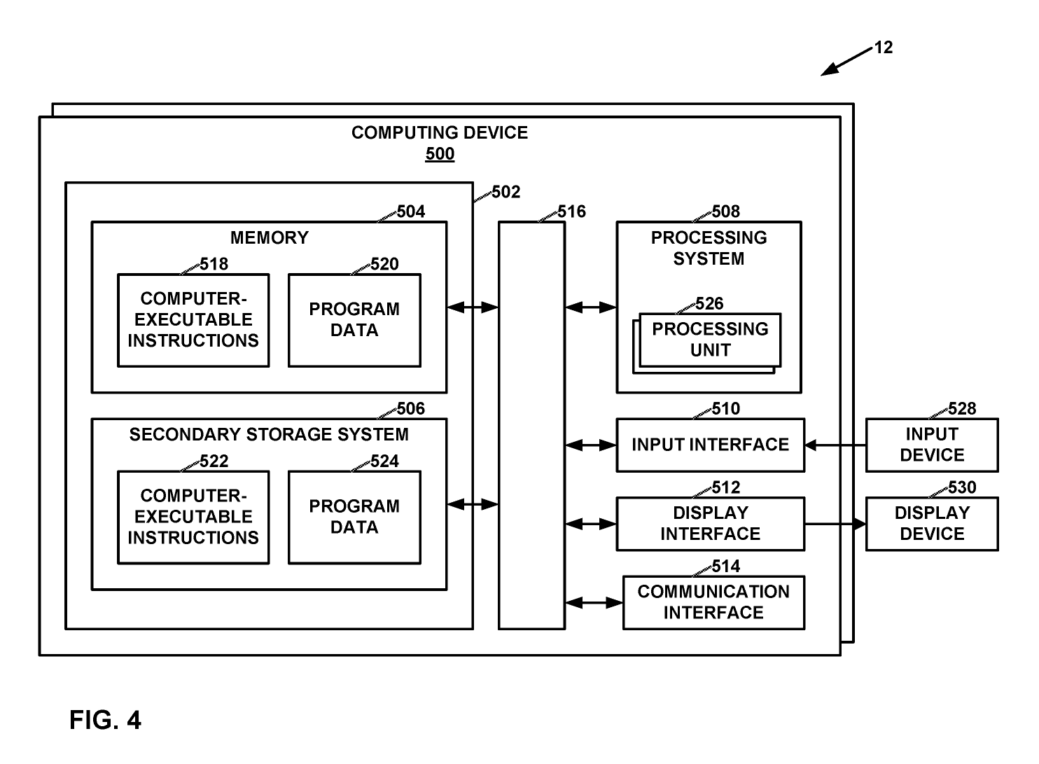

FIG. 4 is a block diagram of an example configuration of computing system 102, which may be configured to implement the techniques of this disclosure. In the example of FIG. 4, computing system 102 comprises a computing device 500 and one or more other computing devices. Moreover, computing system 102 may represent a type of computing device used by patients 108 or clinicians 104.

Computing device 500 is a physical device that processes information. In the example of FIG. 4, computing device 500 comprises a data storage system 502, a memory 504, a secondary storage system 506, a processing system 508, an input interface 510, a display interface 512, a communication interface 514, and one or more communication media 516. Communication media 516 may enable data communication between processing system 508, input interface 510, display interface 512, communication interface 514, memory 504, and secondary storage system 506. Computing device 500 may include components in addition to those shown in the example of FIG. 4. Furthermore, some computing devices do not include all of the components shown in the example of FIG. 4.

A computer system-readable medium may comprise a medium from which a processing system can read data. Computer system-readable media may include computer system storage media and communications media. Computer system storage media may include physical devices that store data for subsequent retrieval. Computer system storage media are not transitory (i.e., non-transitory). For instance, computer system storage media do not exclusively comprise propagated signals. Computer system storage media may include volatile storage media and non-volatile storage media. Example types of computer system storage media may include random-access memory (RAM) units, read-only memory (ROM) devices, solid state memory devices, optical discs (e.g., compact discs, DVDs, Blu-ray discs, etc.), magnetic disk drives, electrically-erasable programmable read-only memory (EEPROM), programmable read-only memory (PROM), magnetic tape drives, magnetic disks, and other types of devices that store data for subsequent retrieval. Communication media may include media over which one device can communicate data to another device. Example types of communication media may include communication networks, communications cables, wireless communication links, communication buses, and other media over which one device is able to communicate data to another device.

Data storage system 502 may comprise a system that stores data for subsequent retrieval. In the example of FIG. 4, data storage system 502 comprises memory 504 and secondary storage system 506. Memory 504 and secondary storage system 506 may store data for later retrieval. In the example of FIG. 4, memory 504 stores computer-executable instructions 518 and program data 520. Furthermore, in the example of FIG. 4, secondary storage system 506 stores computer-executable instructions 522 and program data 524. Physically, memory 504 and secondary storage system 506 may each comprise one or more computer system storage media. Database 110 may be stored in data storage system 502.

In the example of FIG. 4, processing system 508 is coupled to data storage system 502. Processing system 508 may read computer-executable instructions from data storage system 502 and may execute the computer-executable instructions. Execution of the computer-executable instructions by processing system 508 may configure and/or cause computing device 500 to perform the actions indicated by the computer-executable instructions. For example, execution of the computer-executable instructions by processing system 508 can configure and/or cause computing device 500 to provide Basic Input/Output Systems (BIOS), operating systems, system programs, application programs, or may configure and/or cause computing device 500 to provide other functionality. Furthermore, execution of the computer-executable instructions by processing units 526 may cause computing system 102 to provide the functionality ascribed in this disclosure to computing system 102.

Processing system 508 may read the computer-executable instructions from one or more computer system-readable media. For example, processing system 508 may read and execute computer-executable instructions 518 and 522 stored on memory 504 and secondary storage system 506.

Processing system 508 may comprise one or more processing units 526. Processing units 526 may comprise physical devices that execute computer-executable instructions. Processing units 526 may comprise various types of physical devices that execute computer-executable instructions. For example, one or more of processing units 526 may comprise a microprocessor, a processing core within a microprocessor, a digital signal processor, a graphics-processing unit, or another type of physical device that executes computer-executable instructions.

Input interface 510 may enable computing device 500 to receive input from an input device 528. Input device 528 may comprise a device that receives input from a user. Input device 528 may comprise various types of devices that receive input from users. For example, input device 528 may comprise a keyboard, a touch screen, a mouse, a microphone, a keypad, a joystick, a brain-computer system interface device, or another type of device that receives input from a user. In some examples, input device 528 is integrated into a housing of computing device 500. In other examples, input device 528 is outside a housing of computing device 500. In some examples, input device 528 may receive input of quantitative data used in generating the various user interfaces described in this disclosure for facilitating a decision-making process regarding reduction of one or more barriers to patients accessing medical therapies from a healthcare provider.

Display interface 512 may enable computing device 500 to display output on a display device 530. Display device 530 may be a device that presents output. Example types of display devices include printers, monitors, touch screens, display screens, televisions, and other types of devices that display output. In some examples, display device 530 is integrated into a housing of computing device 500. In other examples, display device 530 is outside a housing of computing device 500. In some examples, display device 530 may present the different user interfaces as described above.

Communication interface 514 may enable computing device 500 to send and receive data over one or more communication media. Communication interface 514 may comprise various types of devices. For example, communication interface 514 may comprise a Network Interface Card (NIC), a wireless network adapter, a Universal Serial Bus (USB) port, or another type of device that enables computing device 500 to send and receive data over one or more communication media.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in a computer system-readable medium, such as a computer system-readable storage medium, containing instructions. Instructions embedded or encoded in a computer system-readable medium, including a computer system-readable storage medium, may cause one or more programmable processors, or other processors, to implement one or more of the techniques described herein, such as when instructions included or encoded in the computer system-readable medium are executed by the one or more processors. Computer system readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer system readable media. In some examples, an article of manufacture may comprise one or more computer system-readable storage media.

Various examples have been described. These and other examples are within the scope of the following claims.

What is claimed is:

1. A method for increasing accuracy of a computing system in correlating values of variables in management protocols with health outcomes, the method comprising:
receiving, by a computing system, an indication of user input indicating whether a clinician accepts use of default values for a set of one or more variables in a management protocol (MP) template as values of the variables in an MP for a particular patient based on the MP template, the MP for the particular patient being a protocol the particular patient is advised to follow as part of the particular patient managing a medical condition, each of the variables including a respective action description that contains a space filled by the value of the variable;
generating, by the computing system, a description of the MP for the particular patient, the description of the MP for the particular patient specifying the values of the variables in the MP for the particular patient;
providing, by the computing system, the description of the MP for the particular patient to the particular patient;
after providing the description of the MP for the particular patient to the particular patient, receiving, by the computing system, data indicating health outcomes of the particular patient;
generating, by the computing system, based on the health outcomes of the particular patient and health outcomes of one or more additional patients managing the same medical condition, comparison data comparing health outcomes of patients whose MPs specify a first set of values of the variables versus health outcomes of patients whose MPs specify a second, different set of values of the variables;
outputting, by the computing system, the comparison data for display; and
after outputting the comparison data for display, receiving, by the computing system, an indication of user input indicating updates to the default values for the variables in the MP template.

2. The method of claim 1, further comprising:
prior to receiving the indication of user input indicating whether the clinician accepts the use of the default values in the MP template as the values of the variables in the MP for the particular patient, presenting, by the computing system, a list of the default values for the variables.

3. The method of claim 1, wherein, based on the indication of user input indicating that the clinician does not accept use of the default values for the variables in the MP template as the values of the variables in the MP for the particular patient:
the method further comprises outputting, by the computing system, for display, a user interface comprising user interface features for selecting custom values as the values of the variables in the MP for the particular patient, and
generating the description of the MP for the particular patient comprises generating, by the computing system, the description of the MP for the particular patient such that the description of the MP for the particular patient specifies the custom values for one or more of the variables in the MP for the particular patient.

4. The method of claim 3, wherein outputting the user interface comprises:
automatically setting, by the computing system, values in the user interface features to custom values previously selected by the clinician.

5. The method of claim 1, wherein generating the comparison data comprises:
generating, by the computing system, data indicating whether an incidence of a positive health outcome is greater when the first set of values is used as opposed to the second set of values.

6. The method of claim 1, wherein the clinician is a first clinician and the default values for the set of variables are a first set of group-level default values specific to a first group of clinicians, the method further comprising:
receiving, by the computing system, an indication of user input indicating whether a second clinician in a second group of clinicians accepts use of a set of group-level default values specific to the second group of clinicians as values of the variables in an MP for a second patient based on the MP template, the set of group-level default values specific to the second group of clinicians having one or more values different from values of the variables in the first set of group-level default values.

7. A computing system comprising:
one or more processing circuits configured to:
receive an indication of user input indicating whether a clinician accepts use of default values for a set of variables in a management protocol (MP) template as values of the variables in an MP for a particular patient based on the MP template, the MP for the particular patient being a protocol the particular patient is advised to follow as part of the particular patient managing a medical condition, each of the variables including a respective action description that contains a space filled by the value of the variable;
generate a description of the MP for the particular patient, the description of the MP for the particular patient specifying the values of the variables in the MP for the particular patient;
provide the description of the MP for the particular patient to the particular patient;
after providing the description of the MP for the particular patient to the particular patient, receive data indicating health outcomes of the particular patient;
generate, based on the health outcomes of the particular patient and health outcomes of one or more additional patients managing the same medical condition, comparison data comparing health outcomes of patients whose MPs specify a first set of values of the variables versus health outcomes of patients whose MPs specify a second, different set of values of the variables;
output the comparison data for display; and
after outputting the comparison data for display, receive an indication of user input indicating updates to the default values for the variables in the MP template.

8. The computing system of claim 7, wherein the one or more processing circuits are further configured to:
prior to receiving the indication of user input indicating whether the clinician accepts the use of the default values in the MP template as the values of the variables in the MP for the particular patient, present a list of the default values for the variables.

9. The computing system of claim 7, wherein the one or more processors are configured such that, based on the indication of user input indicating that the clinician does not accept use of the default values in the MP template as the values of the variables in the MP for the particular patient, the one or more processors:
output, for display, a user interface comprising user interface features for selecting custom values for the one or more variables in in the MP for the particular patient, and
generate the description of the MP for the particular patient such that the description of the MP for the particular patient specifies the custom values for the variables in the MP for the particular patient.

10. The computing system of claim 9, wherein the one or more processors are configured such that, as part of outputting the user interface, the one or more processors:
automatically set values in the user interface features to custom values previously selected by the clinician.

11. The computing system of claim 7, wherein the one or more processors are configured such that, as part of generating the comparison data, the one or more processors:
generate data indicating whether an incidence of a positive health outcome is greater when first set of values is used as opposed to the second set of values.

12. The computing system of claim 7, wherein the clinician is a first clinician and the default values for the set of variables are a first set of group-level default values specific to a first group of clinicians, the one or more processors further configured to:
receive an indication of user input indicating whether a second clinician in a second group of clinicians accepts use of a set of group-level default values specific to the second group of clinicians as values of the variables in an MP for a second patient based on the MP template, the set of group-level default values specific to the second group of clinicians having one or more values different from values of the variables in the first set of group-level default values.

13. A non-transitory computer-readable data storage medium having instructions stored thereon that, when executed, configure a computing system to:
receive an indication of user input indicating whether a clinician accepts use of default values for a set of variables in a management protocol (MP) template as values of the variables in an MP for a particular patient based on the MP template, the MP for the particular patient being a protocol the particular patient is advised to follow as part of the particular patient managing a medical condition, each of the variables including a respective action description that contains a space filled by the value of the variable;
generate a description of the MP for the particular patient, the description of the MP for the particular patient specifying the values of the variables in the MP for the particular patient;
provide the description of the MP for the particular patient to the particular patient;
after providing the description of the MP for the particular patient to the particular patient, receive data indicating health outcomes of the particular patient;
generate, based on the health outcomes of the particular patient and health outcomes of one or more additional patients managing the same medical condition, comparison data comparing health outcomes of patients whose MPs specify a first set of values of the variables versus health outcomes of patients whose MPs specify a second, different set of values of the variables;
output the comparison data for display; and
after outputting the comparison data for display, receive an indication of user input indicating updates to the default values for the variables in the MP template.

14. The non-transitory computer-readable data storage medium of claim 13, wherein execution of the instructions further configures the computing system to:
prior to receiving the indication of user input indicating whether the clinician accepts the use of the default values in the MP template as the values of the variables in the MP for the particular patient, present a list of the default values for the variables.

15. The non-transitory computer-readable data storage medium of claim 13, wherein execution of the instructions configures the computing system such that, based on the indication of user input indicating that the clinician does not accept use of the default values in the MP template as the values of the variables in the MP for the particular patient, the computing system:
outputs, for display, a user interface comprising user interface features for selecting custom values for the one or more variables in in the MP for the particular patient, and
generates the description of the MP for the particular patient such that the description of the MP for the particular patient specifies the custom values for the variables in the MP for the particular patient.

16. The non-transitory computer-readable data storage medium of claim 15, wherein execution of the instructions configures the computing system such that, as part of configuring the computing system to output the user interface, execution of the instructions causes the computing system to:
automatically set values in the user interface features to custom values previously selected by the clinician.

17. The non-transitory computer-readable data storage medium of claim 13, wherein execution of the instructions configures the computing system such that, as part of generating the comparison data, the computing system:
generates data indicating whether an incidence of a positive health outcome is greater when first set of values is used as opposed to the second set of values.

18. The non-transitory computer-readable data storage medium of claim 13, wherein the clinician is a first clinician and the default values for the set of variables are a first set of group-level default values specific to a first group of clinicians, execution of the instructions further configures the computing system to:
receive an indication of user input indicating whether a second clinician in a second group of clinicians accepts use of a set of group-level default values specific to the second group of clinicians as values of the variables in an MP for a second patient based on the MP template, the set of group-level default values specific to the second group of clinicians having one or more values different from values of the variables in the first set of group-level default values.

19. A computing system comprising:
means for receiving an indication of user input indicating whether a clinician accepts use of default values for a set of variables in a management protocol (MP) template as values of the variables in an MP for a particular patient based on the MP template, the MP for the particular patient being a protocol the particular patient is advised to follow as part of the particular patient managing a medical condition, each of the variables including a respective action description that contains a space filled by the value of the variable;
means for generating a description of the MP for the particular patient, the description of the MP for the particular patient specifying the values of the variables in the MP for the particular patient;
means for providing the description of the MP for the particular patient to the particular patient;
means for receiving, after providing the description of the MP for the particular patient to the particular patient, data indicating health outcomes of the particular patient;
means for generating, based on the health outcomes of the particular patient and health outcomes of one or more additional patients managing the same medical condition, comparison data comparing health outcomes of patients whose MPs specify a first set of values of the variables versus health outcomes of patients whose MPs specify a second, different set of values of the variables;
means for outputting the comparison data for display; and
means for receiving, after outputting the comparison data for display, an indication of user input indicating updates to the default values for the variables in the MP template.
